(19) United States
(12) Patent Application Publication
(10) Pub. No.: US 2009/0017015 A1
(43) Pub. Date: Jan. 15, 2009

(54) Dpp-iv inhibitors for treating neurodegeneration and cognitive disorders

(76) Inventor: Thomas Edward Hughes, Concord, MA (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/589,690

(22) PCT Filed: Feb. 18, 2005
(86) PCT No.: PCT/EP2005/001729

§ 371 Date: Sep. 19, 2006

Related U.S. Application Data
(60) Provisional application No. 60/546,229, filed on Feb. 20, 2004.
(60) Provisional application No. 60/607,902, filed on Sep. 8, 2004.

(51) Int. Cl. A61K 39/395; A61K 31/4439; A61K 31/40; A61K 31/426; A61K 31/403; A61K 31/445; A61K 31/473; A61K 31/55; A61K 31/66; A61K 31/439; A61P 25/00; A61K 38/13; A61K 31/4164; A61K 31/433; A61K 31/47; A61K 31/505; A61K 31/5415; A61K 31/4985; A61K 33/24

(52) U.S. Cl. 424/131.1; 514/343; 514/423; 514/365; 514/412; 514/307; 514/319; 514/297; 514/215; 514/129; 514/295; 514/396; 514/361; 514/313; 514/256; 514/226.5; 514/249; 424/649; 514/9

(57) ABSTRACT

The invention relates to the use of a Dipeptidyl peptidase IV inhibitor (DPP-IV inhibitor) or a pharmaceutically acceptable salt thereof for the prevention, delay of progression or the treatment of neurodegenerative disorders, cognitive disorders and for improving memory (both short term and long term) and learning ability.

The invention relates to the use of a Dipeptidyl peptidase IV inhibitor (DPP-IV inhibitor) or a pharmaceutically acceptable salt thereof for the prevention, delay of progression or the treatment of neurodegenerative disorders, cognitive disorders and for improving memory (both short term and long term) and learning ability.

The term “DPP-IV inhibitor” is intended to indicate a molecule that exhibits inhibition of the enzymatic activity of DPP-IV and functionally related enzymes, such as from 1-100% or 20-80% inhibition, and specially preserves the action of substrate molecules, including but not limited to glucagon-like peptide-1, gastric inhibitory polypeptide, peptide histidine methionine, substance P, neuropeptide Y, and other molecules typically containing alanine or proline residues in the second aminoterminal position. Treatment with DPP-IV inhibitors prolongs the duration of action of peptide substrates and increases levels of their intact, undegraded forms leading to a spectrum of biological activities relevant to the disclosed invention.

DPP-IV can be used in the control of glucose metabolism because its substrates include the insulinotropic hormones Glucagon like peptide-1 (GLP-1) and Gastric inhibitory peptide (GIP). GLP-1 and GIP are active only in their intact forms; removal of their two N-terminal amino acids inactivates them. In vivo administration of synthetic inhibitors of DPP-IV prevents N-terminal degradation of GLP-1 and GIP, resulting in higher plasma concentrations of these hormones, increased insulin secretion and, therefore, improved glucose tolerance. For that purpose, chemical compounds are tested for their ability to inhibit the enzyme activity of purified CD26/DPP-IV. Briefly, the activity of CD26/DPP-IV is measured in vitro by its ability to cleave the synthetic substrate Gly-Pro-p-nitroanilide (Gly-Pro-pNA). Cleavage of Gly-Pro-pNA by DPP-IV liberates the product p-nitroanilide (pNA), whose rate of appearance is directly proportional to the enzyme activity. Inhibition of the enzyme activity by specific enzyme inhibitors slows down the generation of pNA. Stronger interaction between an inhibitor and the enzyme results in a slower rate of generation of pNA. Thus, the degree of inhibition of the rate of accumulation of pNA is a direct measure of the strength of enzyme inhibition. The accumulation of pNA is measured with a spectrophotometer. The inhibition constant, Ki, for each compound is determined by incubating fixed amounts of enzyme with several different concentrations of inhibitor and substrate.

In the present context “a DPP-IV inhibitor” is also intended to comprise active metabolites and prodrugs thereof, such as active metabolites and prodrugs of DPP-IV inhibitors. A “metabolite” is an active derivative of a DPP-IV inhibitor produced when the DPP-IV inhibitor is metabolised. A “prodrug” is a compound that is either metabolised to a DPP-IV inhibitor or is metabolised to the same metabolite(s) as a DPP-IV inhibitor.

DPP-IV inhibitors are known in the art. In the following reference is made to representatives of DPP-IV inhibitors:

Preferred DPP-IV inhibitors are described in the following patent applications; WO 02053548 especially compounds 1001 to 1293 and examples 1 to 124, WO 02067918 especially compounds 1000 to 1278 and 2001 to 2159, WO 02066627 especially the described examples, WO 02/068420 especially all the compounds specifically listed in the examples I to LXIII and the described corresponding analogues, even preferred compounds are 2(28), 2(88), 2(119), 2(136) described in the table reporting IC50, WO 02083128 such as in the claims 1 to 5 especially compounds described in examples 1 to 13 and the claims 6 to 10, US 2003096846 especially the specifically described compounds, WO 2004/037181 especially examples 1 to 33 and most preferably the compounds described in the claims 3 to 5, WO 0168603 especially compounds of examples 1 to 109, EP1258480 especially compounds of examples 1 to 60, WO 0181337 especially examples 1 to 118, WO 02083109 especially examples 1A to 1D, WO 030003250 especially compounds of examples 1 to 166, most preferably 1 to 8, WO 03035067 especially the compounds described in the examples, WO 03/035057 especially the compounds described in the examples, US2003216450 especially examples 1 to 450, WO 99/46272 especially compounds of claims 12, 14, 15 and 17, WO 0197808 especially compounds of claim 2, WO 03002553 especially compounds of examples 1 to 33, WO 01/34594 especially the compounds described in the examples 1 to 4, WO 02051836 especially examples 1 to 712, EP1245568 especially examples 1 to 7, EP1258476 especially examples 1 to 32, US 2003087950 especially the described examples, WO 02/076450 especially examples 1 to 128, WO 03000180 especially examples 1 to 162, WO 03000181 especially examples 1 to 66, WO 03004498 especially examples 1 to 33, WO 0302942 especially examples 1 to 68, U.S. Pat. No. 6,482,844 especially the described examples, WO 0155105 especially the compounds listed in the examples 1 and 2, WO 0202560 especially examples 1 to 166, WO 03004496 especially examples 1 to 103, WO 03/024965 especially examples 1 to 54, WO 0303727 especially examples 1 to 209, WO 0368757 especially examples 1 to 88, WO 03074500 especially examples 1 to 72, examples 4.1 to 4.23, examples 5.1 to 5.10, examples 6.1 to 6.30, examples 7.1 to 7.23, examples 8.1 to 8.10, examples 9.1 to 9.30, WO 02038541 especially examples 1 to 53, WO 02062764 especially examples 1 to 293, preferably the compound of example 95 (2-{{3-(Aminomethyl)-4-butoxy-2-neopentyl-1-oxo-1,2 dihydro-6-isoquinolinyl}oxy}acetamide hydrochloride), WO 02308090 especially examples 1-1 to 1-109, examples 2-1 to 2-9, example 3, examples 4-1 to 4-19, examples 5-1 to 5-39, examples 6-1 to 6-4, examples 7-1 to 7-10, examples 8-1 to 8-8, examples 7-1 to 7-7 of page 90, examples 8-1 to 8-59 of pages 91 to 95, examples 9-1 to 9-33, examples 10-1 to 10-20, US 2003225102 especially compounds 1 to 115, compounds of examples 1 to 121, preferably compounds a) to z), aa) to az), ba) to bz), ca) to cz) and da) to dk), WO 0214271 especially examples 1 to 320, and US 2003096857, WO 2004/052850 especially the specifically described compounds such as examples 1 to 42 and compounds of claim 1, DE 102 56 264 A1 especially the described compounds such as examples 1 to 181 and the compounds of claim 5, WO 04/076433 especially the compounds specifically described, such as listed in table A, preferably the compounds listed in table B, preferably compounds I to XXXXVII, or compounds of claims 6 to 49, WO 04/071454 especially the specifically described compounds e.g. compounds 1 to 53 or compounds of tables Ia to If, or compounds of claims 2 to 55, WO 02/068420 especially the compounds specifically described, such as the compounds I to LXIII or Beispiele I and analogues 1 to 140 or Beispiele 2 and analogues 1 to 174 or Beispiele 3 and analogues 1, or Beispiele 4 to 5, or Beispiele 6 and analogues 1 to 5, or Beispiele 7 and analogues 1-3, or Beispiele 8 and analogue 1, or Beispiele 9, or Beispiele 10 and analogues 1 to 531 even preferred are compounds of claim 13, WO 03/000250 especially the compounds specifically described, such as the compounds 1 to 166, preferably compounds of examples 1 to 9, WO 03/024942 especially the compounds specifically described, such compounds 1 to 59, compounds of table 1 (1 to 68), compounds of claims 6, 7, 8, 9, WO 03024965024942 especially the compounds specifically described, such compounds 1 to 54, Wo03002593 especially the compounds specifically described, such compounds table 1 or of claims 2 to 15, WO03037327 especially the compounds specifically described, such compounds of examples 1 to 209 WO 03/000250 especially the compounds specifically described, such as the compounds 1 to 166, preferably compounds of examples 1 to 9, WO 03/024942 especially the compounds specifically described, such compounds 1 to 59, compounds of table 1 (1 to 68), compounds of claims 6, 7, 8, 9, WO 03024965024942 especially the compounds specifically described, such compounds 1 to 54, Wo03002593 especially the compounds specifically described, such compounds table 1 or of claims 2 to 15, WO03037327 especially the compounds specifically described, such compounds of examples 1 to 209, WO0238541, WO0230890.

WO 03/000250 especially the compounds specifically described, such as the compounds 1 to 166, preferably compounds of examples 1 to 9, WO 03/024942 especially the compounds specifically described, such compounds 1 to 59, compounds of table 1 (1 to 68), compounds of claims 6, 7, 8, 9, WO 03024965 especially the compounds specifically described, such compounds 1 to 54, Wo03002593 especially the compounds specifically described, such compounds table 1 or of claims 2 to 15, WO03037327 especially the compounds specifically described, such compounds of examples 1 to 209, WO0238541 especially the compounds specifically described, such compounds of examples 1 to 53, WO 03/002531 especially the compounds specifically described preferably the compounds listed on page 9 to 13, most preferably the compounds of examples 1 to 46 and even preferred compound of example 9, U.S. Pat. No. 6,395,767 preferably compound of examples 1 to 109 most preferably compound of example 60, U.S. application Ser. No. 09/788,173 filed Feb. 16, 2001 (attorney file LA50) especially the described examples, WO99/38501 especially the described examples, WO99/46272 especially the described examples and DE19616 486 A1 especially val-pyr, val-thiazolidide, isoleucyl-thiazolidide, isoleucyl-pyrrolidide, and fumar salts of isoleucyl-thiazolidide and isoleucyl-pyrrolidide.

Further preferred DPP-IV inhibitors include the specific examples disclosed in U.S. Pat. Nos. 6,124,305 and U.S. Pat. No. 6,107,317, International Patent Applications, Publication Numbers WO 95153 09 and WO 9818763.

Published patent application WO 9819998 discloses N—(N′-substituted glycyl)-2-cyano pyrrolidines, in particular 1-[2-[5-Cyanopyridin-2-yl]amino]-ethylamino]acetyl-2-cyano-(S)-pyrrolidine (NVP-DPP728) and (2S)-1-[(2S)-2 amino-3,3-dimethylbutanoyl]-2-pyrrolidinecarbonitrile.

In a further preferred embodiment, the DPP-IV inhibitor is a N-peptidyl-O-aroyl hydroxylamine or a pharmaceutically acceptable salt thereof. Aroyl is, for example, naphthylcarbonyl; or benzoyl which is unsubstituted or mono- or disubstituted, for example, by lower alkoxy, lower alkyl, halogen or, preferably, nitro. The peptidyl moiety comprises preferably two α-amino acids, e.g. glycine, alanine, leucine, phenylalanine, lysine or proline, of which the one attached directly to the hydroxylamine nitrogen atom is preferably proline.

DPP-IV inhibitors are in each case generically and specifically disclosed e.g. in WO 98/19998, DE19616 486 A1, WO 00/34241, WO 95/15309, WO 01/72290, WO01/52825, WO03/002553, WO 9310127, WO 99/61431, WO 9925719, WO 9938501, WO 9946272, WO 9967278 and WO 9967279.

In each case in particular in the compound claims and the final products of the working examples, the subject matter of the final products, the pharmaceutical preparations and the claims are hereby incorporated into the present application by reference to these publications.

WO 9819998 discloses N—(N′-substituted glycyl)-2-cyano pyrrolidines, in particular 1-[2-[5-Cyanopyridin-2-yl]amino]-ethylamino]acetyl-2-cyano-(S)-pyrrolidine.

Preferred compounds described in WO03/002553 are listed on pages 9 to 11 and are incorporated into the present application by reference.

DE19616 486 A1 discloses val-pyr, val-thiazolidide, isoleucyl-thiazolidide, isoleucyl-pyrrolidide, and fumar salts of isoleucyl-thiazolidide and isoleucyl-pyrrolidide.

Published patent application WO 0034241 and published patent U.S. Pat. No. 6,110,949 disclose N-substituted adamantyl-amino-acetyl-2-cyano pyrrolidines and N-(substituted glycyl)-4-cyano pyrrolidines respectively. DPP-IV inhibitors of interest are specially those cited in claims 1 to 4. In particular these applications describe the compound 1-[[(3-Hydroxy-1-adamantyl) amino]acetyl]-2-cyano-(S)-pyrrolidine (also known as LAF237 or vildagliptin).

WO 9515309 discloses amino acid 2-cyanopyrrolidine amides as inhibitors of DPP-IV and WO 9529691 discloses peptidyl derivates of diesters of alpha-aminoalkylphosphonic acids, particularly those with proline or related structures. DPP-IV inhibitors of interest are specially those cited in Table 1 to 8.

In WO 01/72290 DPP-IV inhibitors of interest are specially those cited in example 1 and claims 1, 4, and 6.

WO01/52825 specially discloses (S)-1-(2-[5-cyanopyridin-2-yl)amino]ethyl-aminoacetyl)-2-cyano-pyrrolidine or (S)-1-[(3-hydroxy-1 adamantyl)amino]acetyl-2-cyano-pyrrolidine.

WO 9310127 discloses proline boronic esters useful as DPP-IV inhibitors. DPP-IV inhibitors of interest are specially those cited in examples 1 to 19.

Published patent application WO 9925719 discloses sulphostin, a DPP-IV inhibitor prepared by culturing a Streptomyces microorganism.

WO 9938501 discloses N-substituted 4- to 8-membered heterocyclic rings. DPP-IV inhibitors of interest are specially those cited in claims 15 to 20.

WO 9946272 discloses phosphoric compounds as inhibitors of DPP-IV. DPP-IV inhibitors of interest are specially those cited in claims 1 to 23.

Other preferred DPP-IV inhibitors are the compounds of formula I, II or III disclosed in the patent application WO 03/057200 on page 14 to 27. Most preferred DPP-IV inhibitors are the compounds specifically described on pages 28 and 29.

In a further preferred embodiment, the DPP-IV inhibitor is a N-peptidyl-O-aroyl hydroxylamine or a pharmaceutically acceptable salt thereof. Aroyl is, for example, naphthylcarbonyl; or benzoyl which is unsubstituted or mono- or disubstituted, for example, by lower alkoxy, lower alkyl, halogen or, preferably, nitro. The peptidyl moiety comprises preferably two α-amino acids, e.g. glycine, alanine, leucine, phenylalanine, lysine or proline, of which the one attached directly to the hydroxylamine nitrogen atom is preferably proline.

Published patent applications WO 9967278 and WO 9967279 disclose DPP-IV prodrugs and inhibitors of the form A-B-C where C is either a stable or unstable inhibitor of DPP-IV.

Preferably, the N-peptidyl-O-aroyl hydroxylamine is a compound of formula VII

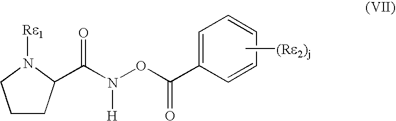

wherein
j is 0, 1 or 2;
Rε₁ represents the side chain of a natural amino acid; and
Rε₂ represents lower alkoxy, lower alkyl, halogen or nitro;
or a pharmaceutically acceptable salt thereof.

In a very preferred embodiment of the invention, the N-peptidyl-O-aroyl hydroxylamine is a compound of formula VIIIa

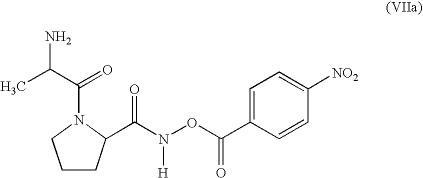

or a pharmaceutically acceptable salt thereof.

N-Peptidyl-O-aroyl hydroxylamines, e.g. of formula VII or VIIa, and their preparation are described by H. U. Demuth et al. in J. Enzyme Inhibition 1988, Vol. 2, pages 129-142, especially on pages 130-132.

Preferred DPP-IV inhibitors are N-substituted adamantyl-amino-acetyl-2-cyano pyrrolidines, N (substituted glycyl)-4-cyano pyrrolidines, N—(N′-substituted glycyl)-2-cyanopyrrolidines, N-aminoacyl thiazolidines, N-aminoacyl pyrrolidines, L-allo-isoleucyl thiazolidine, L-threo-isoleucyl pyrrolidine, and L-allo-isoleucyl pyrrolidine, 1-[2-[(5-cyanopyridin-2-yl)amino]ethylamino]acetyl-2-cyano-(S)-pyrrolidine and pharmaceutical salts thereof.

Preferred DPP-IV inhibitors are those described by Mona Patel and col. (Expert Opinion Investig Drugs. 2003 April; 12(4):623-33) on the paragraph 5, especially P32/98, K-364, FE-999011, BDPX, NVP-DDP-728 and others, which publication is hereby incorporated by reference especially the described DPP-IV inhibitors.

FE-999011 is described in the patent application WO 95/15309 page 14, as compound No. 18.

Another preferred inhibitor is the compound BMS-477118 disclosed in WO 2001068603 or U.S. Pat. No. 6,395,767 (compound of example 60) also known as is (1S,3S,5S)-2-[(2S)-2-amino-2-(3-hydroxytricyclo[3.3.1.1^{3,7}]dec-1-yl)-1 oxoethyl]-2-azabicyclo[3.1.0]hexane-3-carbonitrile, benzoate (1:1) as depicted in Formula M of the patent application WO 2004/052850 on page 2, and the corresponding free base, (1S,3S,5S)-2-[(2S)-2-amino-2-(3-hydroxy-tricyclo[3.3.1.1^{3,7}]dec-1-yl)-1-oxoethyl]-2-azabicyclo-[3.1.0]hexane-3-carbonitrile (M′) and its monohydrate (M″) as depicted in Formula M of the patent application WO 2004/052850 on page 3. The compound BMS-477118 is also known as saxagliptin.

Another preferred inhibitor is the compound GSK23A disclosed in WO 03/002531 (example 9) also known as (2S,4S)-1-((2R)-2-Amino-3-[(4-methoxybenzyl)sulfonyl]-3-methylbutanoyl)-4-fluoropyrrolidine-2-carbonitrile hydrochloride.

P32/98 (CAS number 251572-86-8) also known as 3-[(2S,3S)-2-amino-3-methyl-1-oxopentyl]thiazolidine can be used as 3-[(2S,3S)-2-amino-3-methyl-1-oxopentyl]thiazolidine and (2E)-2-butenedioate (2:1) mixture and is described in WO 99/61431 and the below formula,

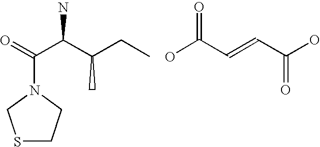

is described in WO 99/61431 and also in Diabetes 1998, 47, 1253-1258, in the name of Probiodrug, as well as the compound P93/01 described by the same company.

Other very preferred DPP-IV inhibitors are the compounds disclosed in the patent application WO 02/083128 such as in the claims 1 to 5. Most preferred DPP-IV inhibitors are the compounds specifically described by the examples 1 to 13 and the claims 6 to 10.

Other very preferred DPP-IV inhibitors are the compounds disclosed By Bristol-Myers Squibb such as Saxagliptin (BMS477118).

Other very preferred DPP-IV inhibitors of the invention are described in the International patent application WO 02/076450 (especially the examples 1 to 128) and by Wallace T. Ashton (Bioorganic & Medicinal Chemistry Letters 14 (2004) 859-863) especially the compound 1 and the compounds listed in the tables 1 and 2. The preferred compound is the compound 21e (table 1) of formula:

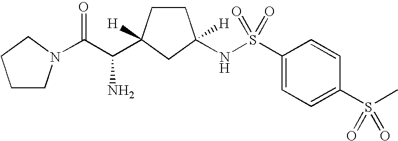

Other preferred DPP-IV inhibitors are described in the patent applications WO 2004/037169 especially those described in the examples 1 to 48 and WO 02/062764 especially the described examples 1 to 293, even preferred are the compounds 3-(aminomethyl)-2-isobutyl-1-oxo-4-phenyl-1,2-dihydro-6-isoquinolinecarboxamide and 2-{[3-(aminomethyl)-2-isobutyl-4-phenyl-1-oxo-1,2-dihydro-6-isoquinolyl]oxy}acetamide described on page 7 and also in the patent application WO2004/024184 especially in the reference examples 1 to 4.

Other preferred DPP-IV inhibitors are described in the patent application WO 03/004498 especially examples 1 to 33 and most preferably the compound of the formula

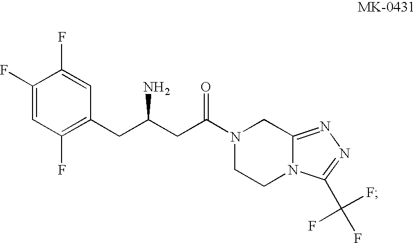

described by the example 7 and also known as MK-0431.

In each case in particular in the compound claims and the final products of the working examples, the subject matter of the final products, the pharmaceutical preparations and the claims are hereby incorporated into the present application by reference to these publications.

Preferred DPP-IV inhibitors are also described in the patent application WO 2004/037181 especially examples 1 to 33 and most preferably the compounds described in the claims 3 to 5.

Preferred DPP-IV inhibitors are N-substituted adamantyl-amino-acetyl-2-cyano pyrrolidines, N (substituted glycyl)-4-cyano pyrrolidines, N—(N′-substituted glycyl)-2-cyanopyrrolidines, N-aminoacyl thiazolidines, N-aminoacyl pyrrolidines, L-allo-isoleucyl thiazolidine, L-threo-isoleucyl pyrrolidine, and L-allo-isoleucyl pyrrolidine, 1-[2-[(5-cyanopyridin-2-yl)amino]ethylamino]acetyl-2-cyano-(S)-pyrrolidine, MK-431 and pharmaceutical salts thereof.

Most preferred DPP-IV inhibitors are selected from [S]-1-[2-(5-cyano-2-pyridinylamino)ethylamino]acetyl-2-pyrrolidine carbonitrile monohydrochloride, (S)-1-[(3-hydroxy-1-adamantyl)amino]acetyl-2-cyano-pyrrolidine and L-threo-isoleucyl thiazolidine (compound code according to Probiodrug: P32/98 as described above), MK-0431, 3-(aminomethyl)-2-isobutyl-1-oxo-4-phenyl-1,2-dihydro-6-isoquinolinecarboxamide and 2-{[3-(aminomethyl)-2-isobutyl-4-phenyl-1-oxo-1,2-dihydro-6-isoquinolyl]oxy}acetamide and optionally pharmaceutical salts thereof.

[S]-1-[2-(5-cyano-2-pyridinylamino)ethylamino]acetyl-2-pyrrolidine carbonitrile monohydrochloride and (S)-1-[(3-hydroxy-1-adamantyl)amino]acetyl-2-cyano-pyrrolidine are specifically disclosed in Example 3 of WO 98/19998 and Example 1 of WO 00/34241, respectively. The DPP-IV inhibitor P32/98 (see above) is specifically described in Diabetes 1998, 47, 1253-1258. [S]-1-[2-(5-cyano-2-pyridinylamino)ethylamino]acetyl-2-pyrrolidine carbonitrile monohydrochloride and (S)-1-[(3-hydroxy-1-adamantyl)amino]acetyl-2-cyano-pyrrolidine can be formulated as described on page 20 of WO 98/19998 or in WO 00/34241.

Especially preferred are 1-{2-[(5-cyanopyridin-2-yl)amino]ethylamino}acetyl-2-(S)-cyano-pyrrolidine (also named [S]-1-[2-(5-cyano-2-pyridinylamino)ethylamino]acetyl-2-pyrrolidine carbonitrile monohydrochloride), of formula:

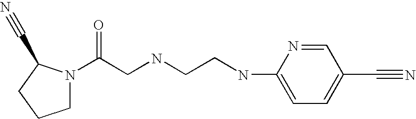

especially the dihydrochloride and monohydrochloride form thereof, pyrrolidine, 1-[(3-hydroxy-1-adamantyl)amino]acetyl-2-cyano-, (S) (also named (S)-1-[(3-hydroxy-1-adamantyl)amino]acetyl-2-cyano-pyrrolidine, LAF237 or vildagliptin) of formula

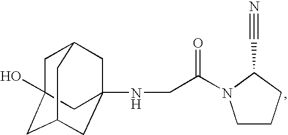

and L-threo-isoleucyl thiazolidine (compound code according to Probiodrug: P32/98 as described above), MK-0431, GSK23A, saxagliptin, 3-(aminomethyl)-2-isobutyl-1-oxo-4-phenyl-1,2-dihydro-6-isoquinolinecarboxamide and 2-{[3-(aminomethyl)-2-isobutyl-4-phenyl-1-oxo-1,2-dihydro-6-isoquinolyl]oxy}acetamide and optionally in any case pharmaceutical salts thereof.

DPP728 and LAF237 are specifically disclosed in Example 3 of WO 98/19998 and Example 1 of WO 00/34241, respectively. The DPP-IV inhibitor P32/98 (see above) is specifically described in Diabetes 1998, 47, 1253-1258. DPP728 and LAF237 can be formulated as described on page 20 of WO 98/19998 or in WO 00/34241, or in the International Patent Application No. EP2005/000400 (application number).

Any of the substances disclosed in the above mentioned patent documents or scientific publications, hereby included by reference, are considered potentially useful as DPP-IV inhibitors to be used in carrying out the present invention.

DPP-IV inhibitor to be used alone according to the present invention can be used in association with a carrier.

A carrier in the instant context is a tool (natural, synthetic, peptidic, non-peptidic) for example a protein which transports specific substances through the cell membrane in which it is embedded and into the cell. Different carriers (natural, synthetic, peptidic, non-peptidic) are required to transport different substances, as each one is designed to recognize only one substance, or group of similar substances.

Any means of detection known by the person skilled in the art can be used to detect the association of the DPP-IV with a carrier, for example, by labelling the carrier.

The DPP-IV inhibitor can be a peptidic or, preferably, non-peptidic one.

Most preferred are orally active DPP-IV inhibitors and pharmaceutical salts thereof.

The active ingredients or pharmaceutically acceptable salts thereof according to the present invention may also be used in form of a solvate, such as a hydrate or including other solvents, used for crystallization. The active ingredients can as well be in any crystalline form.

It has now been surprisingly found that DPP-IV inhibitors are useful for the prevention, delay of progression or the treatment of neurodegenerative disorders, cognitive disorders and for improving memory (both short term and long term) and learning ability.

Preferably the neurodegenerative disorder is selected from conditions and diseases like dementia (e.g. senile dementia, pre-senile dementia (also known as mild cognitive impairment), Alzheimer related dementia (Alzheimer type dementia)), Huntington's chorea, tardive dyskinesia, hyperkinesias, mania, Morbus Parkinson, steel-Richard syndrome, Down's syndrome, myasthenia gravis, nerve and brain trauma, vascular amyloidosis, cerebral haemorrhage with amyloidosis, brain inflammation, Friedrich's ataxia, acute confusion disorders and especially those in which apoptotic necrocytosis plays a part, such as amyotrophic lateral sclerosis, glaucoma and especially Alzheimer's disease.

More preferably the neurodegenerative disorder is selected from Alzheimer's disease and dementia, preferably senile dementia, mild cognitive impairment or Alzheimer type dementia

More preferably the neurodegenerative disorder is Alzheimer's disease.

The use of DPP-IV inhibitor for treating multiple sclerosis, migraine, stroke, cerebral ischemia, and Parkinson's disease was already described in the patent application WO 03/002596, however, unexpected advantages and improved results are obtained when the DPP-IV inhibitor is vildagliptin. The effect on multiple sclerosis can be assed by the protocol of example 13 of WO 03/002596, incorporated herewith by reference.

Thus the present invention also concerns the use of vildagliptin or a pharmaceutical salts thereof, for the manufacture of a medicament for the prevention, delay of progression or the treatment of multiple sclerosis, migraine, stroke, ischemia, cerebral ischemia, ischemic injury and Parkinson's disease.

The present invention relates furthermore to a method for the prevention, delay of progression or the treatment of multiple sclerosis, migraine, stroke, ischemia, cerebral ischemia, ischemic injury and Parkinson's disease comprising administering to a warm-blooded animal, including man, in need thereof, a therapeutically effective amount of vildagliptin or a pharmaceutical salts thereof.

The present invention also concerns the use of vildagliptin or a pharmaceutical salts thereof, for the manufacture of a medicament for the prevention, delay of progression or treatment of general peripheral neuropathies or diabetic peripheral neuropathies. The present invention relates furthermore to a method for the prevention, delay of progression or the treatment of general peripheral neuropathies or diabetic peripheral neuropathies comprising administering to a warm-blooded animal, including man, in need thereof, a therapeutically effective amount of vildagliptin or a pharmaceutical salts thereof.

The invention also provides a use or a method of treating age-related cognitive decline or mild cognitive impairment comprising administering to a patient in need thereof a therapeutically-effective amount of a DPP-IV inhibitor (preferably vildagliptin) as defined above. In a particular embodiment, the invention also provides a method of preventing, retarding or arresting any further age-related cognitive decline or progression of mild cognitive impairment comprising administering to a patient in need thereof a therapeutically-effective amount of a DPP-IV inhibitor (preferably vildagliptin) as defined above or a pharmaceutically acceptable salt thereof.

In a particular embodiment, the invention provides a use or method for preventing or delaying the onset of dementia associated with Alzheimer's disease in a patient with age related cognitive decline or in a patent with mild cognitive impairment.

In a particularly preferred embodiment, the neurodegenerative disorder is selected from Alzheimer's. Preferably for preventing or delaying the onset of Alzheimer's disease (AD) in a patient suffering from age-related cognitive decline or mild cognitive impairment. In a particular embodiment of the invention, the DPP-INV inhibitor is administered to a patient suffering from age-related cognitive decline or mild cognitive impairment who additionally possesses one or more risk factors for developing AD selected from: a family history of the disease; a genetic predisposition to the disease; elevated serum cholesterol; adult-onset diabetes mellitus; raised CSF levels of total tan; raised CSF levels of phospho-tau; and lowered CSF levels of Ap42.

Preferably the cognitive disorder is selected from conditions and diseases like cognitive deficits associated with schizophrenia, age-induced memory impairment, cognitive deficits associated with psychosis, cognitive impairment associated with diabetes, cognitive deficits associated with post-stroke, memory defects associated with hypoxia, cognitive and attention deficits associated with senile dementia, attention-deficit disorders, memory problems associated with mild cognitive impairment, impaired cognitive function associated with dementias, impaired cognitive function associated with Alzheimer's disease, impaired cognitive function associated with Parkinson's disease, impaired cognitive function associated with vascular dementia, cognitive problems associated with brain tumors, Pick's disease, cognitive deficits due to autism, cognitive deficits post electroconvulsive therapy, cognitive deficits associated with traumatic brain injury, amnesic disorders, deliriums, dementias. Cognitive disorder also include, but are not limited to, disorders of learning acquisition (learning disorders), memory consolidation, retrieval memory and retention disorders.

More preferably the cognitive disorder is selected from cognitive impairment associated with diabetes, impaired cognitive function associated with Alzheimer's disease, impaired cognitive function associated with Parkinson's disease, cognitive deficits associated with post-stroke, cognitive and attention deficits associated with senile dementia, memory problems associated with mild cognitive impairment.

In a particularly preferred embodiment the cognitive disorder is selected from cognitive impairment associated with diabetes and impaired cognitive function associated with Alzheimer's disease, cognitive deficits associated with post-stroke.

In a particularly preferred embodiment the cognitive disorder is selected from age-related cognitive decline. Preferably “age-related” refers to a patient aged 55 or over, 65 or over, 75 or over.

DPP-IV inhibitors may also be useful for improving memory (both short term and long term) and learning ability in general such as treating and/or preventing memory impairment in general. For instance, DPP-IV inhibitors can particularly be used to improve learning speed and potential in educational and rehabilitation contexts. In a preferred aspect, DPP-IV inhibitors may be useful for treating impaired memory or learning which is age-associated, is consequent upon electro-convulsive therapy or which is the result of brain damage caused, for example, by stroke, ananesthetic accident, head trauma, hypoglycemia, carbon monoxide poisoning, lithium intoxication or a vitamin deficiency.

The present invention thus concerns the use of a DPP-IV inhibitor or pharmaceutically acceptable salt thereof, for the manufacture of a medicament for the prevention, delay of progression or the treatment of neurodegenerative disorders, cognitive disorders and for improving memory (both short term and long term) and learning ability.

In particular, the present invention relates to a new use of (S)-1-[(3-hydroxy-1-adamantyl) amino]acetyl-2-cyano-pyrrolidine (LAF237 or vildagliptin) or pharmaceutically acceptable salt thereof, for the manufacture of a medicament for the prevention, delay of progression or the treatment of neurodegenerative disorders, cognitive disorders and for improving memory (both short term and long term) and learning ability.

The present invention relates furthermore to a method for the prevention, delay of progression or the treatment of neurodegenerative disorders, cognitive disorders and for improving memory (both short term and long term) and learning ability comprising administering to a warm-blooded animal, including man, in need thereof, a therapeutically effective amount of a DPP-IV inhibitor preferably vildagliptin.

In a further embodiment the present invention relates to use of a DPP-IV inhibitor preferably (S)-1-[(3-hydroxy-1-adamantyl)amino]acetyl-2-cyano-pyrrolidine (LAF237 or vildagliptin) of formula (I) for treating and/or preventing memory or learning impairment, e.g., due to toxicant exposure, brain injury, brain aneurysm, age-associated memory impairment, mild cognitive impairment, epilepsy, mental retardation in children, and dementia resulting from a disease, such as Parkinson's disease, Alzheimer's disease, AIDS, head trauma, Huntington's disease, Pick's disease, Creutzfeldt-Jakob disease, and stroke. In addition, the compounds of the invention may be useful in enhancing memory in normal individuals.

In a further embodiment, the present invention relates to a pharmaceutical composition comprising a therapeutically effective amount of a DPP-IV inhibitor in combination with one or more pharmaceutically acceptable carriers for the prevention, delay of progression or the treatment of neurodegenerative disorders, cognitive disorders and for improving memory (both short term and long term) and learning ability.

It has also been surprisingly found that DPP-IV inhibitors preferably have an effect in neurite growth in neurodegenerative diseases, especially for Alzheimer's and Parkinson's diseases.

The term “prevention” means prophylactic administration of the combination to healthy patients to prevent the outbreak of the conditions mentioned herein. Moreover, the term “prevention” means prophylactic administration of such combination to patients being in a pre-stage of the conditions, to be treated.

The term “delay of progression” used herein means administration of the combination, such as a combined preparation or pharmaceutical composition, to patients being in a pre-stage of the condition to be treated in which patients a pre-form of the corresponding condition is diagnosed.

By the term “treatment” is understood the management and care of a patient for the purpose of combating the disease, condition, or disorder.

Though the causes may differ, patients with neurodegenerative disorders are likely to show localized to generalized atrophy of brain cells leading to compromises in both mental and physical functions.

The condition mediated by DPP-IV is preferably selected from the group consisting of dementia, Alzheimer's disease, amyotrophic lateral sclerosis and glaucoma.

Most preferably, the condition mediated by DPP-IV is dementia, Alzheimer's or Parkinson's disease.

The term “dementia” as used herein includes Alzheimer type dementia, Parkinson type dementia, Huntington type dementia, Pick's type dementia, Creutzfeldt-Jakob type dementia, senile dementia, pre-senile dementia, idiopathic-related dementia, trauma-related dementia, stroke-related dementia, cranial bleed-related dementia, vascular dementia, and includes acute, chronic or recurring forms.

Alzheimer's disease, is the most common form of dementia. This neurological disorder attacks the brain and results in cognitive problems, such as memory loss, impaired thinking, difficulty performing familiar tasks, disorientation to time and place, poor or decreased judgment, problems with language, changes in mood or behaviour and personality.

Advancing age is the single greatest risk factor for Alzheimer's, a disease that strikes 10 percent of individuals by the time they reach age 65 and up to 50 percent by age 85. Part of this increased risk seems to occur because brain cells become increasingly vulnerable to stress as they age.

In Alzheimer's disease, beta-amyloid (Aβ) protein fragments accumulate to form abnormal structures called amyloid plaques. Developing effective Alzheimer treatments requires a thorough understanding of how the disease kills brain cells by disrupting cellular chemistry and communication. Many experts believe that one key to Alzheimer cell death lies in abnormal processing of amyloid precursor protein (APP).

Parkinson's disease is a progressive disorder of the central nervous system, and affecting over 1 million people in the United States. Clinically, the disease is characterized by a decrease in spontaneous movements, gait difficulty, postural instability, rigidity and tremor. Parkinson's disease is caused by the degeneration of the pigmented neurons in the Substantia Nigra of the brain, resulting in decreased dopamine availability.

Parkinson disease affects both men and women. The frequency of the disease is considerably higher in the over 50-age-group, even though there is an alarming increase of patients of younger age. In consideration of the increased life expectancy in this country and worldwide, an increasing number of people will be victims of Parkinson's disease.

Genetically programmed degeneration of neurons in certain areas of the brain cause Huntington's disease. Early symptoms of Huntington's disease include mood swings, or trouble learning new things or remembering a fact. Most drugs used to treat the symptoms of Huntington's disease have side effects such as fatigue, restlessness, or hyperexcitability. Currently, there is no treatment to stop or reverse the progression of Huntington's disease. Therefore, there is a need of a pharmaceutical agent to address the symptoms with fewer side effects.

Diabetes mellitus, like cognitive impairment, is an age-related condition. In the United States, the prevalence of diabetes among people over 65 years is nearly four times as great as that for younger populations. Over 3 million older adults in the United States have been diagnosed with diabetes; another 6 million have undiagnosed diabetes or impaired glucose tolerance (Kenny et al, 1995). Given the combined high prevalence of diabetes and dementia among older adults, even a moderate association between diabetes and cognitive decline could have major public health implications. In addition, both diabetes mellitus and dementia are complex clinical diagnoses whose management poses challenges for clinicians. Evidence increasingly suggests that diabetes is associated with cognitive impairment. Moreover, cognitive impairment may influence self-care and thereby complicate diabetes management. The effect of diabetes on cognitive function appears to be due to factors intrinsic to diabetes (glycemia and insulin resistance), diabetes-related complications (stroke), or unwanted side effects from diabetes management (hypoglycemia). Thus, there is a need for new drugs that provide improvement in treating and preventing cognitive impairment in diabetic patients.

Alzheimer's disease has many aspects, including cognitive and attention deficits. Its diagnosis is described in the Diagnostic and Statistical Manual of Mental Disorders' 4th ea. published by the American Psychiatric Association SM-T) (e.g. pages 139-143). Diagnostic criteria for Alzheimer's disease includes the development of multiple cognitive deficits in a patient manifested by (1) memory impermanent (impaired ability to learn new information or to recall previously learned information), and (2) one (or more) of the following cognitive disturbances (a) aphasia (language disturbance), (b) apraxia (impaired ability to carry out motor activities despite intact motor function), (c) agnosia (failure to recognize or identify objects despite intact sensory function) and (d) disturbances in executing functioning (i.e. planning, organizing, sequencing, abstracting). Currently, these deficits are treated with cholinesterase inhibitors. These inhibitors slow the break down of acetylcholine, and thereby provide a general nonspecific increase in the activity of the cholinergic nervous system. Since the drugs are nonspecific, they have a wide variety of side effects. Thus, there is a need for new drugs that provide improvement in the cognitive and attention deficits associated with Alzheimer's disease without the side effects created by nonspecific stimulation of the cholinergic pathways.

Tardive dyskinesia is associated with the use of conventional antipsychotic drugs. This disease is characterized by involuntary movements most often manifested by puckering of the lips and tongue and/or writhing of the arms or legs. The incidence of tardive dyskinesia is about 5% per year of drug exposure among patients taking conventional antipsychotic drugs. In about 2% of persons with the disease, tardive dyskinesia is severely disfiguring. Currently, there is no generalized treatment for tardive dyskinesia. Furthermore, the removal of the effect-causing drugs is not always an option due to underlying problems. Therefore, there is a need for a pharmaceutical agent to address the symptoms of tardive dyskinesia

Pre-senile dementia (mild cognitive impairment) concerns memory impairment rather than attention deficit problems and otherwise unimpaired cognitive functioning. Mild cognitive impairment is distinguished from senile dementia in that mild cognitive impairment involves a more persistent and troublesome problem of memory loss for the age of the patient. There currently is no medication specifically identified for treatment of mild cognitive impairment, due somewhat to the newness of identifying the disease. Therefore, there is a need for a drug to treat the memory problems associated with mild cognitive impairment.

Age-related cognitive decline and mild cognitive impairment (MCI) are conditions in which a memory deficit is presents but other diagnostic criteria for dementia are absent (Santacruz and Swagerty, American Family Physician, 63 (2001), 703-13). (See also “The ICD-10 Classification of Mental and Behavioural Disorders”, Geneva: World Health Organization, 1992, 64-5). When used herein, age-related cognitive decline is characterized by a decline of at least four preferably six months' duration in at least one of: memory and learning; attention and concentration, thinking; language; and visuospatial functioning and a score of more than one standard deviation below the noun on standardized neuropsychologic testing such as the MMSE. In particular, there may be a progressive decline in memory. In the more severe condition mild cognitive impairment (MCI), the degree of memory impairment is outside the range considered normal for the age of the patient but Alzheimer's disease (AD) is not present. The differential diagnosis of MCI and mild AD is described by Petersen et al., Arch. Neurol., 56 (1999), 303-8. In the same article, Petersen et al. disclose that the patients suffering from MCI typically experience a progressive increase in cognitive impairment and in many cases develop AD. Further information on the differential diagnosis of MCI is provided by Knopman et al, Mayo Clinic Proceedings, 78 (2003), 1290-1308: In a study of elderly subjects, TuokLo et al (Arch, Neurol., 60 (2003) 577-82) found that those exhibiting MCI at the outset had a three-fold increased risk of developing dementia within 5 years.

Grundman et al (J. Mol. Neurosci., 19 (2002), 23-28) report that lower baseline hippocampal volume in MCI patients is a prognostic indicator for subsequent AD.

Similarly, Andreasen et al (Aeta Neurol. Scand, 107 (2003) 47-51) report that high CSF levels of total tau high CSF levels of phospho-tau and lowered CSF levels of A,B42 are all associated with increased risk of progression from MCI to AD.

Age-related cognitive decline and mild cognitive impairment are distinct from the significant cognitive deficit that sometimes results from cerebral or systemic diseases and traumas, such as stroke, concussion, or major disjunction of the pituitary.

Persons with Down's syndrome have in all or at least some of their cells an extra, critical portion of the number 21 chromosome. Adults who have Down's syndrome are known to be at risk for Alzheimer-type dementia. Currently, there is no proven treatment for Down's syndrome. Therefore, there is a need to address the dementia associated with Down's syndrome.

Senile dementia is not a single disease state. However, the conditions classified under this name frequently include cognitive and attention deficits. Generally, these deficits are not treated. Accordingly, there is a need for a drug that provides improvement in the cognitive and attention deficits associated with senile dementia.

Pick's disease results from a slowly progressive deterioration of social skills and changes in personality with the resulting symptoms being impairment of intellect, memory, and language. Common symptoms include memory loss, lack of spontaneity, difficulty in thinking or concentrating, and speech disturbances. Currently, there is no specific treatment or cure for Pick's disease but some symptoms can be treated with cholinergic and serotonin-boosting antidepressants. In addition, antipsychotic medications may alleviate symptoms in FTD patients who are experiencing delusions or hallucinations. Therefore, there is a need for a pharmaceutical agent to treat the progressive deterioration of social skills and changes in personality and to address the symptoms with fewer side effects.

Traumatic brain injury occurs when the brain is damaged from a sudden physical assault on the head. Symptoms of the traumatic brain injury include confusion and other cognitive problems. Therefore, there is a need to address the symptoms of confusion and other cognitive problems.

Brain tumors are abnormal growths of tissue found inside of the skull. Symptoms of brain tumors include behavioral and cognitive problems. Surgery, radiation, and chemotherapy are used to treat the tumor, but other agents are necessary to address associated symptoms. Therefore, there is a need to address the symptoms of behavioral and cognitive problems.

The pharmaceutical activities as effected by administration of representatives of the class of DPP-IV inhibitors used according to the present invention can be demonstrated e.g. by using corresponding pharmacological models known in the pertinent art. The person skilled in the pertinent art is fully enabled to select a relevant animal test model to prove the herein before and hereinafter indicated therapeutic indications and beneficial effects.

The pharmacological activity of the compounds and combinations according to the invention in neurodegenerative disease may, for example, be established in:
A) In the observation test in the mouse the compounds at doses from 0.01 to 100 mg/kg, more preferred doses ranged from 0.1 to 50 mg/kg p.o. provoke a prolongation of the wake phase and an increased reactivity to external stimuli,
B) In the sleep/wake cycle test in chronically implanted rats the compound doses from about 0.01 to 100 mg/kg, more preferred doses ranged from 0.1 to 50 mg/kg p.o. increase the REM sleep phase, and
C) In the carbon-14 deoxyglucose rat test (according to the principles of L:Sokoloff, journal of cerebral Blood flow and metabolism 1981, 1, 7-36, H. E Savaki et al., Brain research 1982, 233, 347 and J. Mc Culloch et al., Journal of cerebral Blood Flow and Metabolism 1981, 1, 133-136), the compounds at doses from about 0.01 to 100 mg/kg, more preferred doses ranged from 0.1 to 50 mg/kg p.o. increase the carbon-14 deoxyglucose uptake in particular areas of the brain, particularly the limbic system.

In the sleep/wake cycle of the long-term implanted rat (for the method, see J-M Vigouret et al., J. pharmacology 10, 503 (1978)), the compounds according to the invention when administered at doses from 0.01 to 100 mg/kg, more preferred doses ranged from 0.1 to 50 mg/kg p.o. (oral administration) can effect an increase in vigilance by prolonging the wakes phases.

Moreover after administration of doses from 0.01 to 100 mg/kg, more preferred doses ranged from 0.1 to 50 mg/kg p.o. to rats with bilateral lesion of the locus coeruleus (LC) and the Nuccleus basalis Meynert (NBM), the compound according to the invention can improve significantly the cognitive performance as measured by the ability to avoid an electric shock in the shuttle box.

The method is similar to that described by V. Haroutunian et al. in Bran Research 507 (1990) 261-266. Male OFA rats (300 g) are anaesthetzed with pentobarbital and positioned in a stereotaxic apparatus with the upper incisor bar set 5 mm (LC) or 3.3 mm (NBM) below the interaural line. The lesions are carried out with a radio frequency lesion generator at 60 c during 10 seconds. 5 week after lessoning, behavioural testing is performed, using the active avoidance test in the shuttle box as described by A. R. Dravid, A-L. Jaton and E. B. Van Deusen in experimental Brain Research, Suppl. 13, p 249 (1986).

The invention is also based on the surprising discovery that compounds and combinations, according to the invention exhibit a pronounced protective action on facial motor neurons against apoptotic necrocytosis, and can be assed at doses from 0.01 to 100 mg/kg, more preferred doses ranged from 0.1 to 50 mg/kg s.c. and below administered to newborn rats in an experimental procedure according to Ausari et al., J. Neuroscience 13, 4042-4053 (1993), and exhibit a pronounced protective action on hippocampus pyramidal cells for a period of 4 days against necrocytosis caused by the administration of kainic acid, which can be assed after the administration of doses from 0.01 to 100 mg/kg, more preferred doses ranged from 0.1 to 50 mg/kg s.c. and below to fully grown rats in an experimental procedure according to Golowitz and Paterson, Soc. Neurosc. Abstr. 20, 246, 113.2 (1994).

A major rat models in Alzheimer disease have contributed to the understanding of neurodegenerative disease and have yielded promising new approaches to treatment.

Mutations in the amyloid precursor protein (APP) gene cause early-onset familial Alzheimer disease (AD) by affecting the formation of the amyloid β (Aβ) peptide, the major constituent of AD plaques. Transgenic mice have been generated, expressing APP with mutations in codons 717 and 670/671, using several neuron-specific promotors to drive expression of human APP cDNAs. The degree of pathology depends on expression levels and specific mutations. A 2-fold overexpression of human APP with the Swedish double mutation at positions 670/671 combined with the V7171 mutation causes Aβ deposition in neocortex and hippocampus of 18-month-old transgenic mice.

APP23 transgenic mice, over express one mutant human amyloid precursor protein, and exhibit one hallmark of Alzheimer's disease pathology, namely the extracellular deposition of amyloid plaques (Calhoun et al., Proceedings of the National Academy of Sciences of The United States of America (1999 Nov. 23), vol 96 No 24, 14088-14093)).

This APP23 mice model, in addition to amyloid plaques develops cerebrovascular accumulation of amyloid β. The cerebral amyloid angiopathy and associated pathologies in this mouse exhibited a striking similarity to that observed in aged individuals and AD patients. The cerebrovascular accumulation of amyloid β (CAA) in APP23 mice led to focal neuron loss, dystrophic synaptic buttons, and activation of microglia, providing evidence that CAA leads to neurodegeneration.

This APP23 mice model is particularly useful to show the pharmacological activity of the compounds and combinations according to the invention.

The examples 2-4 described in the patent application WO2005009349 describe other protocols to assess the activity of the compounds and combinations of the present invention to treat or prevent, X syndrome, AD, Parkinson's' disease

The pharmacological activity of the compounds and combinations according to the invention in improving cognitive function may, for example, be assessed using tests known to a person skilled in the art such as standardized psychometric tests (e.g. Wechsler Memory Scale, the Wechsler Adult Intelligence Scale, Raven's Standard Progressive Matrices, Schaie-Thurstone Adult Mental Abilities Test), neuropsychological tests (e.g. Luria-Nebraska), metacognitive self-evaluations (e.g. Metamemory Questionnaire), visual-spatial screening tests (e.g. Poppelreuter's Figures, Clock Recognition, Honeycomb Drawing and Cancellation), cognitive screening tests (e.g. Folstein's Mini Mental State Test) and reaction time tests. Such standardized tests as listed above are described in Ruoppila, I. and Suutama, T. (1997) Scand. J. Soc. Med. Suppl. 53, 44-65 and serve as examples, said reference is incorporated in its entirety herein. The term “cognitive function” includes the functions assessed by any such test.

A clinical protocol to show the positive effect of a DPP-4 inhibitor or the combination of the invention AD disease development is described in the patent application WO 2004/082706 on pages 31-37, which is incorporated herein by reference.

The patient's degree of cognitive decline or impairment is also advantageously assessed at regular intervals before during and/or after a course of treatment with the DPP-IV inhibitor or a pharmaceutically acceptable salt thereof so that changes therein may be detected e.g. the slowing or halting of cognitive decline variety of neuropsychological tests are known in the art for this purpose, such as the Mini Mental State Examination (MMSE) with norms adjusted for age and education (Folstein et al., J. Psych. Res., 12 (1975), 196-198, Anthony et al., Psychological Med. 12 (1982), 397-408; Cockrell et al., Psychopharmacology, 24 (1988), 689-692; Crum et al., J. Am. Med. Assoc'n. 18 (1993), 2386-2391). The MMSE is a brief, quantitative measure of cognitive status in adults. It can be used to screen for cognitive decline or impairment, to estimate the severity of cognitive decline or impairment at a given point in time, to follow the course of cognitive changes in an individual over time, and to document an individual's response to treatment.

Other standard tests for cognitive performance e.g. the Alzheimer's Disease Assessment Scale (ADAS-cog) are described by Doraiswamy (Neurology. 1997 June; 48(6):1511-7) and in the patents US20040024043 and U.S. Pat. No. 6,369,046. The ADAS-cog is a multi-item instrument for measuring cognitive performance which include elements of memory, orientation, retention, reasoning, language and praxis. US20040024043 describes also an in vivo test model in rodents in example 5 and a clinical Study Design in example 9. Another clinical Study Design is described by U.S. Pat. No. 6,369,046 (example 1).

A further useful in vivo protocol which can be used to show that DPP-IV inhibitors can improving cognitive function is described in the European Patent No. 1310258 (examples 5-8).

WO2004004664, WO2004004702, WO2003101276 (examples 1-9) or WO2004096225 in example 3 describe protocols which can be applied to assess the advantages of vildagliptin for the treatment or prevention of ischemia or ischemic injury.

WO0110867 describes protocols which can be applied to assess the advantages of vildagliptin for the treatment or prevention of stokes.

WO 2004006911 (experimental part), WO 0162277 (example 2) provide protocols to which can be applied to assess the advantages of vildagliptin for the treatment or prevention of peripheral neuropathies, especially diabetic peripheral neuropathies. The topical administration can be replaced by oral administration of vildagliptin. Peripheral sensory neuropathy is a relatively frequent and often debilitating complication of diabetes mellitus (Greene et al, 1990) but its aetiology and underlying pathophysiology are uncertain (Ward, 1992).

The above mentioned documents especially the described test models are hereby incorporated by reference.

Another aspect of the invention relates to a combination of at least one DPP-IV inhibitor or a pharmaceutically acceptable salt thereof with at least one COMBINATION PARTNER OF THE INVENTION which can be used for the prevention, delay of progression or the treatment of neurodegenerative disorders, cognitive disorders and for improving memory (both short term and long term) and learning ability to treat warm-blooded animals including mammals, especially humans having or susceptible to neurodegenerative diseases especially Alzheimer or Parkinson disease.

All the more surprising is the experimental finding that the combined administration of a DPP-IV inhibitor especially (S)-1-{2-[5-cyanopyridin-2-yl)amino]ethyl-aminoacetyl}2-cyano-pyrrolidine (DPP728) or (S)-1-[(3-hydroxy-1-adamantyl)amino]acetyl-2-cyano-pyrrolidine (LAF237), and at least one further COMBINATION PARTNER OF THE INVENTION results not only in a beneficial, especially a potentiating or synergistic therapeutic effect but also in additional benefits resulting from combined treatment such as a surprising prolongation of efficacy, a broader variety of therapeutic treatment and surprising beneficial effects on neurodegenerative diseases and conditions and cognitive disorders, e.g. such as the disorders already described herein.

The COMBINATION PARTNER OF THE INVENTION comprises for example drugs belonging to other pharmacological classes of relevance for peripheral and central degenerative diseases, such as anti-inflammatory drugs, antioxidants agents, and neuroprotective agents (e.g. glutamate receptor antagonists) in the case of both peripheral neuropathies and neurodegeneratives diseases, MAO inhibitors, COMT inhibitors, as well as acetylcholine esterase inhibitors (for example rivastigmine (Exelon)), butyrylcholinesterase inhibitors, inhibitors of gamma and beta secretases, inhibitors of amyloid aggregation, dopamine agonist or antagonist, and immunization both active (with amyloid beta peptide conjugated or not to adjuvants) and passive (with specific antibodies to amyloid beta peptide) immunization in the case of Alzheimer's disease, drugs for treating cognitive disorders such as a selective inhibitor of acetylcholinesterase e.g. Donepezil.

The COMT inhibitors are for example, but not limited to, tolcapone and entacapone.

The anti-inflammatory agent, includes, but is not limited to, naproxen sodium, diclofenac sodium, diclofenac potassium, celecoxib, sulindac, oxaprozin, diflunisal, etodolac, meloxicam, ibuprofen, ketoprofen, nabumetone, refecoxib, methotrexate, leflunomide, sulfasalazine, gold salts, RHo-D Immune Globulin, mycophenylate mofetil, cyclosporine, azathioprine, tacrolimus, basiliximab, daclizumab, salicylic acid, acetylsalicylic acid, methyl salicylate, diflunisal, salsalate, olsalazine, sulfasalazine, acetaminophen, indomethacin, sulindac, mefenamic acid, meclofenamate sodium, tolmetin, ketorolac, dichlofenac, flurbinprofen, oxaprozin, piroxicam, meloxicam, ampiroxicam, droxicam, pivoxicam, tenoxicam, phenylbutazone, oxyphenbutazone, antipyrine, aminopyrine, apazone, zileuton, aurothioglucose, gold sodium thiomalate, auranofin, methotrexate, colchicine, allopurinol, probenecid, sulfinpyrazone and benzbromarone or betamethasone and other glucocorticoids.

Preferably the compound having acetylcholine esterase inhibitory activity is selected from the group consisting of 3-[1-(phenylmethyl)-4-piperidinyl]-1-(2,3,4,5-tetrahydro-1H-1-benzazepin-8-yl)-1-propanone fumarate, 2,3-dihydro-5,6-dimethoxy-2-[[1-(phenylmethyl)-4-piperidinyl]methyl]-1H-inden-1-one hydrochloride (donepezil, sold under the trade mark ARICEPT®), (S)-3-[1-(dimethylamino)ethyl]phenyl N-ethyl-N-methylcarbamate (rivastigmine), 9-amino-2,3,5,6,7,8-hexahydro-1H-cyclopenta[b]quinoline (ipidacrine), 1,2,3,4-tetrahydro-9-aminoacridinamine hydrochloride (tacrine, marketed under the trade mark COGNEX®), 8-[3-[4-(diethylcarbamoyl)piperazin-1-yl]propyl]-1,3,7-trimethylxanthine hydrochloride (stacofylline), 4a,5,9,10,11,12-hexahydro-3-methoxy-11-methyl-6-H-benzofuro[3a,3,2-ef][2]benzazepin-6-ol (galantamine), and dimethyl (2.2.2-trichloro-1-hydroxyethyl)phosphonate (metrifonate), eptastigmine, velnacrine, phystostigmine, icozepil, amiridine, minaprine, huperzine, huprine, bis-tetrahydroaminoacridine (bis-THA), imidazoles, 1,2,4-thiadiazolidinone, benzazepine, 4,4′-bipyridine, indenoquinolinylamine, decamethonium, edrophonium, propidium, fasciculins, organophosphates, carbamates, Imino 1,2,3,4-tetrahydrocyclopent[b]indole carbamates, N-Pyrimidine 4-acetylaniline, 7-aryloxycoumarin, propargylamino carbamates, zifrosilone NOS inhibitors, ACh precursors such as choline and pyrrolidinedholine, or cholinergic receptor agonists (e.g. nicotinic, particularly cry, and muscarinic) and therapeutically and pharmaceutically acceptable salts thereof.

Preferably the antioxidants are selected from vitamins C and E, the NMDA modulator is memantine, the MAO inhibitor is selected from rasagiline, selegiline, tranylcypromine iproniazid, clorgyline, phenelzine and isocarboxazid.

In the treatment of AD, standard dosages pf tacrine presently used are 10 mg four times a day, 40 mg/d being the recommended maximum. Presently, capsules of tacrine are taken orally. For donepezil, the standard dosage is 5 mg/d, with a recommended maximum of 10 mg/day. Presently, tablets of donepezil are taken orally.

For rivastigmine, 1.5 mg twice a day is the standard dosage, with a recommended maximum of 6 mg twice a day. Presently, capsules of rivastigmine are taken orally. For galantamine, the standard dosage presently used is 4 mg twice a day. Presently, tablets of galantamine are taken orally.

In a preferred embodiment, tacrine is administered at a dosage of a bout 0.1 to mg per person per day, preferably of about 10 to 150 mg, per person per day, more preferably about 20 to 60 mg per person per day, or about 60 to 100 me per person per day.

In another preferred embodiment, donepezil is administered at a dosage of about 0.1 to 200 mg per person a day, preferably of about 1 to 100 mg per person a day, more preferably about 2 to 30 mg per person a day, or about 30 to 60 mg per person a day.

In another preferred embodiment, rivastigmine is administered at a dosage of about 0.1 to 200 mg per person a day, preferably of about 0.3 to 50 mg per person a day, more preferably about 0.5 to 20 mg per person a day, or about 20 to 40 mg per person a day.

In another preferred embodiment, galantamine is administered at a dosage of about 0.1 to 200 mg per person a day, preferably of about 0.5 to 100 mg per person a day, more preferably about 1 to 30 mg per person a day, or about 30 to 60 mg per person a day.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

In a preferred embodiment, cholinesterase inhibitors are preferably administered orally.

Dopamine agonist or antagonist, are for example, but not limited to, Levodopa, L-DOPA/carbidopa combinations, cocaine, o-methyl-tyrosine, reserpine, tetrabenazine, benzotropine, pargyline, fenodolpam mesylate, cabergoline, pramipexole dihydrochloride, ropinorole, amantadine hydrochloride, selegiline hydrochloride, carlbidopa, pergolide mesylate, Sinemet CR, or Symmekel.

The COMBINATION PARTNER OF THE INVENTION comprises for example other drugs improving cognitive function such as agents directed at modulating GABA, NMDA, cannabinoid, AMPA, kainate, phosphodiesterase (PDE), PKA, PKC, CREB or nootropic systems.

The results of the studies show that the combination according to the present invention can be used for the prevention delay of progression or the treatment of neurodegenerative disorders, cognitive disorders and for improving memory (both short term and long term) and learning ability, in particular the diseases already mentioned above.

The term “synergistic” shall mean that the drugs, when taken together, produce a total joint effect that is greater than the sum of the effects of each drug when taken alone.

The term “potentiation” shall mean an increase of a corresponding pharmacological activity or therapeutical effect, respectively. Potentiation of one component of the combination according to the present invention by co-administration of another component according to the present invention means that an effect is being achieved that is greater than that achieved with one component alone.

The structure of the active agents identified by code nos., generic or trade names may be taken from the actual edition of the standard compendium “The Merck Index” or from databases, e.g. Patents International (e.g. IMS World Publications). The corresponding content thereof is hereby incorporated by reference. Any person skilled in the art is fully enabled to identify the active agents and, based on these references, likewise enabled to manufacture and test the pharmaceutical indications and properties in standard test models, both in vitro and in vivo.

A further aspect of the present invention is the use of a pharmaceutical composition comprising as active ingredients a DPP-IV inhibitor alone or in combination with at least one further COMBINATION PARTNER OF THE INVENTION, in each case in free form or in form of a pharmaceutically acceptable salt thereof for the preparation of a pharmaceutical composition for the prevention, delay of progression or the treatment of neurodegenerative disorders, cognitive disorders and for improving memory (both short term and long term) and learning ability in particular the preferred conditions described above.

The invention also relates to a pharmaceutical composition comprising, as active ingredients a DPP-IV inhibitor alone or in combination with at least one further COMBINATION PARTNER OF THE INVENTION, in each case in free form or in form of a pharmaceutically acceptable salt thereof.

Another aspect of the present invention is the use of a pharmaceutical composition comprising, as active ingredients a DPP-IV inhibitor alone or in combination with at least one further COMBINATION PARTNER OF THE INVENTION, in each case in free form or in form of a pharmaceutically acceptable salt thereof for the preparation of a pharmaceutical composition for the prevention, delay of progression or the treatment of neurodegenerative disorders, cognitive disorders and for improving memory (both short term and long term) and learning ability in particular the preferred conditions described above.

The invention also relates to a method for the prevention, delay of progression or the treatment of neurodegenerative disorders, cognitive disorders and for improving memory (both short term and long term) and learning ability, comprising ad ministering to a warm-blooded animal, including man, in need thereof jointly therapeutically effective amounts of a composition comprising, as active ingredients a DPP-IV inhibitor alone or in combination with at least one further COMBINATION PARTNER OF THE INVENTION, in each case in free form or in form of a pharmaceutically acceptable salt thereof.

These pharmaceutical preparations are for enteral, such as oral, and also rectal or parenteral, administration to homeotherms, with the preparations comprising the pharmacological active compound either alone or together with customary pharmaceutical auxiliary substances. For example, the pharmaceutical preparations consist of from about 0.1% to 90%, preferably of from about 1% to about 80%, of the active compound. Pharmaceutical preparations for enteral or parenteral, and also for ocular, administration are, for example, in unit dose forms, such as coated tablets, tablets, capsules or suppositories and also ampoules. These are prepared in a manner that is known per se, for example using conventional mixing, granulation, coating, solubilizing or lyophilising processes. Thus, pharmaceutical preparations for oral use can be obtained by combining the active compound with solid excipients, if desired granulating a mixture which has been obtained, and, if required or necessary, processing the mixture or granulate into tablets or coated tablet cores after having added suitable auxiliary substances.

The dosage of the active compound can depend on a variety of factors, such as mode of administration, homeothermic species, age and/or individual condition.

In the field of neurodegenerative disease, the preferred patient population age is from 50 years onwards, most preferred from 65 years onwards.

Preferred dosages, for those active ingredients of the pharmaceutical combination according to the present invention that are commercially available, are especially therapeutically effective commercially available dosages.

The dosage of the active compound can depend on a variety pf factors, such as mode of administration, homeothermic species, age and/or individual condition.

The corresponding active ingredient or a pharmaceutically acceptable salt thereof may also be used in form of a hydrate or include other solvents used for crystallization.

For these indications, the exact dosage will of course vary depending upon the compound employed, mode of administration and treatment desired. The compound may be administered by any conventional route, non-oral or preferably orally.

In general, satisfactory results are obtained when administered at a daily dosage of from about 0.01 to 100 mg/kg, more preferred doses ranged from 0.1 to 50 mg/kg.

For the larger mammals, an indicated total daily dosage is in the range from about 0.01 to 100 mg/kg of the compound, conveniently administered in divided doses 2 to 4 times a day in unit dosage form containing for example from about 0.1 to about 50 mg or 100 mg of the compound in sustained release form.

Appropriate daily oral dosage especially for vildagliptin is between 1 and 500 mg preferably between 10 and 100 mg e.g. 10 mg, or between 25 and 100 mg most preferably between 50 and 10, e.g. 25 mg or 40 or 50 or 70 or 100 mg.

Appropriate unit doses for oral administration especially for vildagliptin contain for example about 10 to about 100 mg of the compounds preferably 25 mg or 50 mg or 100 mg.

Appropriate doses for parenteral administration especially for vildagliptin contain for example about 10 to about 50 mg or 25 to about 100 mg of the compound, e.g. 25 mg, 50 mg, 75 mg or 100 mg.

Appropriate unit doses for oral administration especially for preventive treatment contain for example about 0.5 to about 15 mg of the compounds, e.g from 1 to 10 mg. Appropriate doses for parenteral administration contain for example about 0.2 to about 30 mg of the compound, e.g. from 0.3 to 10 mg.

The compounds may be administered in similar manner to known standards for uses in these utilities. The suitable daily dosage for a particular compound will depend on a number of factors such as its relative potency of activity. A person skilled in the pertinent art is fully enabled to determine the therapeutically effective dosage.

The compound of the invention may be administered in free base for or as a pharmaceutically acceptable acid addition or quaternary ammonium salt. Such salts may be prepared in conventional manner and exhibit the same order of activity as the free forms.

If these compounds have, for example, at least one basic center, they can form acid addition salts. Corresponding acid addition salts can also be formed having, if desired, an additionally present basic center. The compounds having an acid group (for example COOH) can also form salts with bases. For example, the compounds to be combined can be present as a sodium salt, as a maleate or as a dihydrochloride. The active ingredient or a pharmaceutically acceptable salt thereof may also be used in form of a hydrate or include other solvents used for crystallization.

It is one objective of this invention to provide a pharmaceutical composition (fixed combination) comprising a (i) DPP-IV inhibitor or a pharmaceutically acceptable salt thereof and (ii) at least one further COMBINATION PARTNER OF THE INVENTION and at least one pharmaceutically acceptable carrier.

The pharmaceutical compositions according to the invention can be prepared in a manner known per se and are those suitable for enteral, such as oral or rectal, and parenteral administration to mammals (warm-blooded animals), including man, comprising a therapeutically effective amount of the pharmacologically active compound, alone or in combination with one or more pharmaceutically acceptable carries, especially suitable for enteral or parenteral application.

In this composition, components (i) and (ii) can be administered together, one after the other or separately in one combined unit dose form or in two separate unit dose forms. In one preferred embodiment of the invention, the unit dose form is a fixed combination. In a fixed combination the components (i) and (ii) are administered in the form of a single galenic formulation, e.g. a single tablet or a single infusion.

The pharmaceutical combination according to the present invention as described hereinbefore and hereinafter may be used for simultaneous use or sequential use in any order, for separate use or as a fixed combination.

Under certain circumstances, drugs with different mechanisms of action may be combined. However, just considering any combination of drugs having different modes of action but acting in the similar field does not necessarily lead to combinations with advantageous effects.

Further benefits are that lower doses of the individual drugs to be combined according to the present invention can be used to reduce the dosage, for example, that the dosages need not only often be smaller but are also applied less frequently, or can be used in order to diminish the incidence of side effects. This is in accordance with the desires and requirements of the patents to be treated.

The pharmaceutical combination according to the present invention comprises a “kit of parts” in the sense that the components can be dosed independently or by use of different fixed combinations with distinguished amounts of the components at different time points. The parts of the “kit of parts” can then e.g. be administered simultaneously or chronologically staggered, that is at different time points and with equal or different time intervals for any part of the “kit of parts”. Preferably, the time intervals are chosen such that the effect on the treated disease or condition in the combined use of the parts is larger than the effect that would be obtained by use of only any one of the components.

The invention furthermore relates to a commercial package comprising the combination according to the present invention together with instructions for simultaneous, separate or sequential use.

A therapeutically effective amount of each of the components of the combination of the present invention may be administered simultaneously or sequentially and in any order, and the components may be administered separately or as a fixed combination. For example, the method of treatment of the invention may comprise (i) administration of a DPP-IV inhibitor in free or pharmaceutically acceptable salt form and (ii) administration of at least one further COMBINATION PARTNER OF THE INVENTION simultaneously or sequentially in any order, in jointly therapeutically effective amounts; preferably in synergistically effective amounts, e.g. in daily dosages corresponding to the ratios described herein.

Further benefits are that lower doses of the individual drugs to be combined according to the present invention can be used to reduce the dosage, for example, that the dosages need not only often be smaller but are also applied less frequently, or can be used in order to diminish the incidence of side effects. This is in accordance with the desires and requirements of the patients to be treated.

The dosage range of the combination of a DPP-IV inhibitor and at least one further COMBINATION PARTNER OF THE INVENTION to be employed depends upon factors known to the person skilled in the art including species of the warm-blooded animal, body weight and age, the nature and severity of the condition to be treated, the mode of administration and the particular substance to be employed. Unless stated otherwise herein, the DPP-IV inhibitor and at least one further COMBINATION PARTNER OF THE INVENTION are preferably divided and administered from one to four times per day.

The invention has been described above by reference to preferred embodiments but, as those skilled in the art will appreciate, many additions, omissions and modifications are possible all within the scope of the claims below.

All patents and literature references cited in this specification are hereby incorporated by reference in their entirety. In case of inconsistencies, the present description, including the definitions and interpretations, will prevail.

### EXPERIMENTAL PART

### Example 1

### Treatment for Preventing or Delaying the Onset of Alzheimer's Disease

One 25 mg or 50 mg tablet of vildagliptin is administered daily with water to subjects in need of such treatment.

### Example 2

### Treatment for Preventing or Delaying the Onset of Alzheimer's Disease in a Subject Exhibiting Mild Cognitive Impairment

A subject having mild cognitive impairment is identified using the MMSE or similar diagnostic tool.

One 25 mg or 50 mg tablet of vildagliptin is administered daily with water to said subject. The cognitive status of the subject is monitored periodically using the MMSE or similar tool, and the subject is monitored for clinical symptoms of dementia.

### Example 3

### Treating, Preventing or Delaying Cognitive Impairment Associated with Diabetes

One 50 mg tablet of vildagliptin is administered daily with water to subjects in need of such treatment e.g. diabetic patients. The cognitive status of the subject is monitored periodically using the MMSE or similar tool.

### Example 4

The ameliorative effect of vildagliptin or the combined use of vildagliptin with the compounds having cholinesterase inhibitory activity on learning deficits is investigated in aged rats. The following methods describe a set of experiments using the vildagliptin as monotherapy or a combination of vildagliptin with a cholinesterase inhibitor e.g. donepezil.

Methods Male (3 to 27 months old) rats of transgenic strain are used. The aged rats are divided into the following four groups (the active ingredient dosage can be adapted by the person skilled in the art).

1) Control group: Repeated administration of placebo pill.
2) Vildagliptin group: Repeated oral administration of vildagliptin 3 mg/kg.
3) Cholinesterase inhibitor group: Repeated oral administration of donepezil 0.3 mg/kg.
4) Combination group: Repeated oral administration of vildagliptin 3 mg/kg and donepezil 0.3 mg/kg.

In the combination group, vildagliptin is administered 30 minutes after administration of donepezil.

Passive avoidance learning test is started on day 14 of treatment, and Morris water maze learning test on day 20 of treatment.

On each day of experiment, vildagliptin and/or a cholinesterase inhibitor are administered 30 minutes and 1 hour, respectively, before initiation of the trial.

1. Passive Avoidance Learning: The passive avoidance learning test is performed using a chamber consisting of light and dark compartments. Young rats (pill, 10 animals) and aged rats (control group, 10 animals; vildagliptin group; 10 animals, donepezil group, 10 animals; combination group, 10 animals) are individually placed in the light compartment and 10 seconds later, the sliding door is opened. After a mouse moves to the dark compartment, the mouse is kept there for about 10 seconds with the door closed. One to two hours after the habituation trial, acquisition trial is performed.

In the acquisition trial, after a mouse moved to the dark compartment, a foot shock (0.4 mA, 3 seconds) is given through the grid floor. Retention trials are performed 24 hours after acquisition trials.

In each trial, the latency from opening of the slide door until the animal moved to the dark compartment (step-through latency) is measured.

2. Morris Water Maze Learning Same animals used in the passive avoidance test are subjected for the water maze task. However, some rats can not swim well in the water tank, thus they are excluded in the water maze task. The water maze learning test is performed on young rats (saline, 10 animals) and aged rats (control group, 9 animals; vildagliptin group, 9 animals: Donepezil group, 8 animals; combination group, 8 animals).

In pretraining which is performed for swimming training and motivation for escaping from water, four trials are performed using a water bath, 80 cm in diameter, in a condition that the platform is visible. From the following day, using a water bath, 120 cm in diameter, learning trials, one session (four trials) per day, are performed with the platform being placed below the water.

### 1. Passive Avoidance Learning

The control group will show a significant decrease in the avoidance time as compared with the young group. The test can accordingly assess that the vildagliptin group and the donepezil group show significant improvement of the learning deficit in aged rats. The test can accordingly assess an improvement of the cognitive status of the treated subject.

This test can also indicate that combination of vildagliptin and donepezil improves the learning deficit in aged rats, and this effect is greater than that seen when either drug is used alone. It can furthermore show that the combination has improved results or advantages than that seen when either drug is used alone.

### 2. Water Maze Learning

In the water maze task, the control group will % how a significant prolongation of latency to find platform submerged in the water compared with the young rats. The test can accordingly assess that vildagliptin group and the donepezil group show a significant improvement in water maze learning deficit. The test can accordingly assess an improvement of the cognitive status of the treated subject.

This test can also indicate that combination of vildagliptin and donepezil improves water maze learning deficit in aged rats, and this effect is greater than that seen when either drug is used alone. It can furthermore show that the combination has improved results or advantages than that seen when either drug is used alone.

Other tests may be performed using animal models e.g. of dementia such as some of those described and reviewed in the following references: Higgins L. S., Vol. Med. Today 1999, 5(6):274-6; Borchelt D. R. et al., Brain Pathol. 1998, 8(4):735-57 and Guenette S. Y. et al., Neurobiol. Aging 1999, 20(2):201-11.

### Example 5

The effects of vildagliptin and the herein described combinations in a model of Parkinson disease are investigated in mice. Male C57/BL6 mice are injected once daily for 7 days with MPTP (30 mg/kg, i.p.). Vildagliptin is administered once or twice daily for 14 days. On day 28, striata are removed, homogenized in perchloric acid, and centrifuged. The supernatant is removed and analyzed for dopamine and other monoamines such as serotonin by reverse-phase HPLC and electrochemical detection. Anti-Parkinson activity is assessed in comparison to the reference compound, e.g. selegiline.

1) A method for the prevention, delay of progression or the treatment of neurodegenerative disorders, cognitive disorders and for improving memory and learning ability, comprising:
administering to a warm-blooded animal, in need thereof, a therapeutically effective amount of a DPP-IV inhibitor.

2) (canceled)

3) A pharmaceutical composition, comprising:
a therapeutically effective amount of a DPP-IV inhibitor in combination with one or more pharmaceutically acceptable carriers for the prevention, delay of progression or the treatment of neurodegenerative disorders, cognitive disorders and for improving memory and learning ability.

4) The method of claim 1, wherein the neurodegenerative disorder is selected from the group consisting of dementia, senile dementia, mild cognitive impairment, Alzheimer related dementia, Huntington's chores, tardive dyskinesia, hyperkinesias, mania, Morbus Parkinson, steel-Richard syndrome, Down's syndrome, myasthenia gravis, nerve and brain trauma, vascular amyloidosis, cerebral haemorrhage with amyloidosis, brain inflammation, Friedrich's ataxia, acute confusion disorders, acute confusion disorders in which apoptotic necrocytosis plays a part, amyotrophic lateral sclerosis, glaucoma, and Alzheimer's disease.

5) The method of claim 1, wherein the neurodegenerative disorder is selected from the group consisting of Alzheimer's disease and dementia, preferably senile dementia, mild cognitive impairment and Alzheimer type dementia.

6) The method of claim 1 for preventing or delaying the onset of dementia associated with Alzheimer's disease in a patient with age related cognitive decline or in a patient with mild cognitive impairment.

7) The method of claim 1 for preventing or delaying the onset of Alzheimer's disease in a patient suffering from age-related cognitive decline or mild cognitive impairment.

8) The method of claim 1, wherein the cognitive disorder is selected from the group consisting of cognitive deficits associated with schizophrenia, age-induced memory impairment, cognitive deficits associated with psychosis, cognitive impairment associated with diabetes, cognitive deficits associated with post-stroke, memory defects associated with hypoxia, cognitive and attention deficits associated with senile dementia, attention-deficit disorders, memory problems associated with mild cognitive impairment, impaired cognitive function associated with dementias, impaired cognitive function associated with Alzheimer's disease, impaired cognitive function associated with Parkinson's disease, impaired cognitive function associated with vascular dementia, cognitive problems associated with brain tumors, Pick's disease, cognitive deficits due to autism, cognitive deficits post electroconvulsive therapy, and cognitive deficits associated with traumatic brain injury, amnesic disorders, deliriums, dementias.

9) The method of claim 1, wherein the cognitive disorder is selected from the group consisting of disorders of learning acquisition, memory consolidation, retrieval memory and retention disorders.

10) The method of claim 1, wherein the cognitive disorder is selected from the group consisting of cognitive impairment associated with diabetes, impaired cognitive function associated with Alzheimer's disease, impaired cognitive function associated with Parkinson's disease, cognitive deficits associated with post-stroke, cognitive and attention deficits associated with senile dementia, and memory problems associated with mild cognitive impairment.

11) The method of claim 1, wherein the cognitive disorder is selected from the group consisting of cognitive impairment associated with diabetes, impaired cognitive function associated with Alzheimer's disease, and cognitive deficits associated with post-stroke.

12) The method of claim 1 to improve learning speed and potential in educational and rehabilitation contexts.

13) The method of claim 1 for treating impaired memory or learning which is age-associated, is consequent upon electro-convulsive therapy or which is the result of brain damage.

14) The method of claim 1 for preventing, retarding or arresting any further age-related cognitive decline or progression of mild cognitive impairment.

15) The method of claim 1 for treating impaired memory or learning which is the result of brain damage caused by; stroke, ananesthetic accident, head trauma, hypoglycemia, carbon monoxide poisoning, lithium intoxication or a vitamin deficiency.

16) The method of claim 1 for treating and/or preventing memory impairment.

17) The method of claim 1 for treating and/or preventing memory impairment due to toxicant exposure, brain injury, brain aneurysm, age-associated memory impairment, mild cognitive impairment, epilepsy, mental retardation in children, and dementia resulting from a disease, such as Parkinson's disease, Alzheimer's disease, AIDS, head trauma, Huntington's disease, Pick's disease, Creutzfeldt-Jakob disease, and stroke.

18) The method of claim 1, wherein the DPP-IV inhibitor is 1-{-2-[(5-cyanopyridin-2-yl)amino]ethylamino}acetyl-2-(S)-cyano-pyrrolidine, vildagliptin, L-threo-isoleucyl thiazolidine, MK-0431, GSK23A, saxagliptin, 3-(aminomethyl)-2-isobutyl-1-oxo-4-phenyl-1,2-dihydro-6-isoquinolinecarboxamide and 2-{[3-(aminomethyl)-2-isobutyl-4-phenyl-1-oxo-1,2-dihydro-6-isoquinolyl]oxy}acetamide, or optionally in any case pharmaceutical salts thereof.

19) The method of claim 1, wherein the DPP-IV inhibitor is vildagliptin or a pharmaceutically acceptable salt thereof.

20) The method of claim 1, wherein the DPP-IV inhibitor is administered in combination with at least one further drug which can be used for the prevention, delay of progression, or treatment of neurodegenerative disorders, cognitive disorders or a drug for improving memory.

21) A pharmaceutical composition comprising
a) a DPP-IV inhibitor or a pharmaceutically acceptable salt thereof and
b) at least one drug which can be used for the prevention, delay of progression or treatment of neurodegenerative disorders, cognitive disorders or a drug for improving memory; and
c) at least one pharmaceutically acceptable carrier.

22) The pharmaceutical composition according to claim 21, wherein the DPP-IV inhibitor is 1-{2-[(5-cyanopyridin-2-yl)amino]ethylamino}acetyl-2-(S)-cyano-pyrrolidine, vildagliptin, L-threo-isoleucyl thiazolidine, MK-0431, GS1423A, saxagliptin, 3-(aminomethyl)-2-isobutyl-1-oxo-4-phenyl-1,2-dihydro-6-isoquinolinecarboxamide and 2-{[3-(aminomethyl)-2-isobutyl-4-phenyl-1-oxo-1,2-dihydro-6-isoquinolyl]oxy}acetamide, or optionally in any case pharmaceutical salts thereof.

23) The pharmaceutical composition according to claim 21, wherein the DPP-IV inhibitor is vildagliptin, or a pharmaceutically acceptable salt thereof.

24) The pharmaceutical composition according to claim 21, wherein the drug which can be used for the prevention, delay of progression or treatment of neurodegenerative disorders, cognitive disorders or a drug for improving memory, is selected from anti-inflammatory drugs, antioxidants agents, neuroprotective agents, glutamate receptor antagonists, acetylcholine esterase inhibitors, butyrylcholinesterase inhibitors, MAO inhibitors, dopamine agonist or antagonist, inhibitors of gamma and beta secretases, inhibitors of amyloid aggregation, amyloid beta peptide, antibodies to amyloid beta peptide, inhibitors of acetylcholinesterase, agents directed at modulating GABA, NMDA, cannabinoid, AMPA, kainate, phosphodiesterase (PDE), PKA, PKC, CREB or nootropic systems.

25) The pharmaceutical composition according to claim 21, wherein the drug which can be used for the prevention, delay of progression or treatment of neurodegenerative disorders, cognitive disorders or the drug for improving memory, is selected from donepezil, rivastigmine, ipidacrine, tacrine, stacofylline, galantamine, metrifonate, eptastigmine, velnacrine, phystostigmine, icozepil, amiridine, minaprine, huperzine, huprine, bis-tetrahydroaminoacridine (bis-THA), imidazoles, 1,2,4-thiadiazolidinone, benzazepine, 4,4′-bipyridine, indenoquinolinylamine, decamethonium, edrophonium, propidium, fasciculins, organophosphates, carbamates, Imino 1,2,3,4-tetrahydrocyclopent[b]indole carbamates, N-Pyrimidine 4-acetylaniline, 7-aryloxycoumarin, propargylamino carbamates, zifrosilone, NOS inhibitors, ACh precursors, choline pyrrolidinedholine, cholinergic receptor agonists, vitamins C and E, memantine, rasagiline, selegiline, tranylcypromine, iproniazid, clorgyline, pheneizine, isocarboxazid, tolcapone and entacapone, naproxen sodium, diclofenac sodium, diclofenac potassium, celecoxib, sulindac, oxaprozin, diflunisal, etodolac, meloxicam, ibuprofen, ketoprofen, nabumetone, refecoxib, methotrexate, leflunomide, sulfasalazine, gold salts, RHo-D Immune Globulin, mycophenylate mofetil, cyclosporine, azathioprine, tacrolimus, basiliximab, daclizumab, salicylic acid, acetylsalicylic acid, methyl salicylate, diflunisal, salsalate, olsalazine, sulfasalazine, acetaminophen, indomethacin, sulindac, mefenamic acid, meclofenamate sodium, tolmetin, ketorolac, dichlofenac, flurbinprofen, oxaprozin, piroxicam, meloxicam, ampiroxicam, droxicam, pivoxicam, tenoxicam, phenylbutezone, oxyphenbutezone, antipyrine, aminopyrine, apezone, zileuton, aurothioglucose, gold sodium thiomalate, auranofin, methotrexate, colchicine, allopurinol, probenecid, sulfinpyrazone and benzbromarone or betamethasone and other glucocorticoids, or pharmaceutically acceptable salts thereof.

26) The pharmaceutical composition according to claim 21, wherein the drug which can be used for the prevention, delay of progression or treatment of neurodegenerative disorders, cognitive disorders or the drug for improving memory, is selected from donepezil, tacrine, rivastigmine, galantamine, vitamin C, vitamin E, memantine, rasagiline, selegiline, tranylcypromine, iproniazid, clorgyline, pheneizine and isocarboxazid.
